(19) United States
(12) Patent Application Publication
(10) Pub. No.: US 2026/0053116 A1
(43) Pub. Date: Feb. 26, 2026

(54) Non-Invasive Pet Health-Monitoring System

(71) Applicant: PRONOVA Inc., Waltham, MA (US)

(72) Inventors: Jaime Paris Meseguer, Waltham, MA (US); Swapnil Tiwari, Medford, MA (US); Amanda Jean Ritz, Tewksbury, MA (US); David Andrew Sopchak, Waltham, MA (US)

(21) Appl. No.: 19/305,967

(22) Filed: Aug. 21, 2025

Related U.S. Application Data
(60) Provisional application No. 63/686,606, filed on Aug. 23, 2024.

(51) Int. Cl. A01K 29/00; B01L 3/00; G16H 50/20

(52) U.S. Cl. CPC A01K 29/005; B01L 3/502707; G16H 50/20; B01L 2200/16; B01L 2300/025; B01L 2300/0636; B01L 2300/0825; B01L 2400/0694

(57) ABSTRACT

A system and method of monitoring health of an animal includes obtaining a fluidic sample from the animal. The fluidic sample is applied to a sensor array that includes a plurality of sensing areas, each sensing area including a sensor for sensing a target analyte, wherein the sensor array includes sensors that sense different target analytes. Input data from at least two sensors from the sensor array that sense different target analytes is received at an interface of a Point of Care Device (PCD). The Point of Care Device (PCD) includes a controller configured to receive the input data from the interface. Quantitative analysis on the fluidic sample is provided based on the input data. Results of the quantitative analysis may be displayed at the Point of Care (POC) device or at a device external the Point of Care (POC) device, such as a smart phone.

### CROSS-REFERENCE TO RELATED APPLICATIONS

This application claims priority from U.S. Provisional Application No. 63/686,606, filed Aug. 23, 2024, which is hereby incorporated herein by reference in its entirety.

### TECHNICAL FIELD

The present invention generally relates to a system and methodology for quantitatively detecting one or more analytes in a sample. More particularly, a test system and methodology based on, for example, enzymes, antibodies, aptamers and/or molecularly imprinted polymers (MIPS), is provided for quantitatively detecting an analyte in a biological fluid. The biological fluid may be, without limitation a saliva sample, e.g., a saliva sample from a subject, such as a pet, for assessing the current overall health conditions of key biological functions. Assessed health conditions may be associated with the heart, kidney, liver, inflammation, metabolic state, stress state and/or other biological functions that can have a negative effect on the overall health of the subject. Trends, patterns, correlations and relationships to established health outcomes can also be generated and/or provided resulting in better care for the subject.

### BACKGROUND ART

Ensuring the ongoing health of companion animals and household pets-especially dogs, cats, and horses-presents a paramount concern for pet owners. The objective is both clear and complex: safeguarding the health and longevity of these animals, often considered as family members. However, a significant challenge presents itself in this endeavor: the inability of pets to articulate their ailments or discomforts, impeding the timely and accurate identification of health issues. Often the only option for the pet owner is a visit to a veterinarian, which may present its own set of challenges related to availably, cost, ease of access and scheduling with work.

This communication barrier remains unbroken primarily due to the intrinsic communication limitations of non-human species. Moreover, many health conditions in pets do not manifest observable symptoms until the conditions have reached advanced stages, necessitating more intensive treatments. “Observable symptoms” refers to recognizable signs of illness or discomfort, such as changes in behavior, eating habits, or physical appearance, while “advanced stages” denote periods when conditions have progressed to a point where treatment becomes more complicated and/or are less likely to succeed.

Furthermore, animals instinctively conceal their symptoms, a survival behavior to prevent appearing weak, which could potentially attract predators. This instinct, combined with diagnostic and health monitoring methods that are often inconvenient, costly, and invasive, culminates in added stress for both pets and owners, leading to infrequent monitoring and delayed diagnoses.

The challenge in addressing this issue lies in balancing several critical factors: the health monitoring method must be non-invasive, ensuring ease of use and the pet's comfort and compliance; it must generate reliable and consistent data for accurate health assessments; it needs to be affordable, encouraging regular monitoring; and it should be user-friendly, accommodating various technical proficiencies among pet owners. Additionally, any effective solution must account for biological diversity among pets, such as breed-specific characteristics, various life stages, and unique health profiles, which add complexity to creating standardized health monitoring solutions.

There are several shortcomings in current available options for pet care. Market offerings primarily cater to professional veterinary use, failing to address the pet owner's need for direct, easy-to-understand health insights and the ability to preemptively act on early signs of discomfort or disease in their pets. Conventional veterinary visits, though thorough, are often once a year or more, offering only episodic insights into a pet's health. These visits can be financially burdensome and often cause anxiety for pets and pet owners alike.

In addition to visiting a veterinarian, pet owners can implement other options, such as wearable health trackers, periodic clinical blood tests, and various home-use health monitoring kits. However, these solutions also fall short. For example, wearables typically only track physical activity, and blood tests are invasive and again require professional settings such as again going to the veterinary clinic. Home-use kits are often overly simplistic, lacking the depth required for comprehensive health assessments (such as urine color-changing strips) or necessitate long waiting periods for lab results, thus failing to offer immediate health insights.

Additionally, home-based urine or fecal tests, while insightful, are sporadic and rely on the stressful collection process and correct handling. DNA tests offer valuable genetic predisposition data but fail to give ongoing health monitoring, are typically costly and don't yield immediate, actionable health strategies. Intrusive solutions, like implants, are in preliminary stages and face resistance due to their invasive nature, potential health risks, and associated high costs.

Real-time health monitoring systems and methods are urgently required that tackle this significant, unresolved challenge in pet health monitoring, mitigating issues related to invasiveness, lack of comprehensive insight, user complexity, and prohibitive costs, and that transcend the constraints of existing solutions by delivering detailed, real-time health data to pet owners.

### SUMMARY OF THE INVENTION

In accordance with an embodiment of the invention, a method of monitoring health of an animal is provided. The method includes obtaining a fluidic sample from the animal. The fluidic sample is applied to a sensor array that includes a plurality of sensing areas, each sensing area including a sensor for sensing a target analyte, wherein the sensor array includes sensors that sense different target analytes. Input data from at least two sensors from the sensor array that sense different target analytes is received at an interface of a Point of Care Device (PCD), the Point of Care Device (PCD) including a controller configured to receive the input data from the interface. Quantitative analysis on the fluidic sample is provided based on the input data. Results of the quantitative analysis are displayed, without limitation, on either the Point of Care (POC) device and/or on an external device/display (e.g., a smart phone) that interfaces with the Point of Care (POC) device.

In accordance with related embodiments of the invention, the sensor array may be positioned on a sensor strip, wherein receiving includes inserting the strip into the interface of the Point of Care Device (PCD). The sensor strip may be a single use, disposable item. In other embodiments, the sensor strip may be reusable.

In accordance with further related embodiments of the invention, applying may include directing the fluidic sample to the plurality of sensing areas, while limiting and/or preventing contamination between the sensing areas. Directing the fluidic sample may include using gravity and/or microfluidics. Directing the fluidic sample may include directing the fluidic sample to the sample areas via channels, each channel including an incline and/or a one-way valve to prevent backflow.

In accordance with still further related embodiments of the invention, the method may further include functionalizing one or more of the sensing areas and/or sensors using a biological recognition element. The biological recognition element may be, without limitation, one of, or a combination of, enzymes, antibodies, aptamers and/or molecularly imprinted polymers (MIPS). The obtained fluidic sample may be mixed with a buffer solution prior to applying the fluidic sample to the sensor array.

In accordance with yet further related embodiments of the invention, data associated with the input data from the Point of Care Device (PCD) may be transmitted to a second device and/or cloud system. Performing the quantitative analysis on the fluidic sample may include: performing the quantitative analysis at the Point of Care Device (PCD), the second device and/or the cloud system; and determining at least one health status of the animal, the health status based on input data from one or more of the sensors. The health status may be based on input data from at least two sensors sensing different target analytes.

In accordance with still further related embodiments of the invention, performing the quantitative analysis on the fluidic sample may be performed solely at the Point of Care Device.

The quantitative analysis may be performed on pure, unprocessed saliva. Performing the quantitative analysis may include applying machine learning to determine at least one health status of the animal. Performing the quantitative analysis includes using input data accrued over a period of time, wherein the period of time is at least one of days and years.

In accordance with another embodiment of the invention, a system for monitoring health of an animal is provided. The system includes a sensor array having a plurality of sensing areas, each sensing area including a sensor for sensing a target analyte, wherein the sensor array includes sensors that sense different target analytes. The system further includes a Point of Care Device (PCD) that includes: an interface that receives input data from at least two sensors from the sensor array that sense different target analytes; a controller configured to receive the input data at the interface and provide quantitative analysis on the fluidic sample based on the sensed target analytes.

In accordance with related embodiments of the invention, the sensor array may be positioned on a sensor strip, wherein the interface of the Point of Care Device (PCD) includes a slot for receiving the sensor. The sensor strip may be a single use, disposable item. In other embodiments, the sensor strip may be reusable.

In accordance with further related embodiments of the invention, the system may include a distributor for directing the fluidic sample to the plurality of sensing areas, while limiting and/or preventing contamination between the sensing areas. Directing by the distributor may include using gravity and/or microfluidics. The distributor may include channels for directing the fluidic sample to particular sensing areas, each channel including an incline and/or a one-way valve to prevent backflow.

In accordance with still further related embodiments of the invention, the controller may include a communication interface for transmitting data associated with the input data to a second device and/or cloud system. The Point of Care Device (PCD), second device and/or cloud may be configured to analyze the input data and provide at least one health status, the at least one health status based on input data from one or more of the sensors. Analyzing the data may include using input data accrued over a period of time, wherein the period of time is at least one of days and years.

The input data may be from at least two sensors sensing different target analytes. The Point of Care Device (PCD), second device and/or cloud system may be configured to apply machine learning in determining the at least one health status. The results of the quantitative analysis may be displayed at the Point of Care Device (PCD), or on a device external to the Point of Care Device (PCD), such as a smart phone.

### BRIEF DESCRIPTION OF THE DRAWINGS

The foregoing features of the invention will be more readily understood by reference to the following detailed description, taken with reference to the accompanying drawings.

FIG. 1 shows a system for monitoring health of an animal, in accordance with an embodiment of the invention.

FIG. 2A shows a schematic of a screen-printed electrode array, in accordance with an embodiment of the invention. FIG. 2B shows an isometric view of the screen-printed electrode array, in accordance with an embodiment of the invention. FIG. 2C shows a screen-printed electrode array in combination with a cover to form wells, in accordance with an embodiment of the invention.

FIG. 3 shows a Point-of-Care Device, in accordance with an embodiment of the invention.

FIG. 4 shows a PCB board for use for a multiplexer potentiostat for the analysis of screen-printed electrode arrays or other electrochemical sensors, in accordance with an embodiment of the invention.

FIG. 5 shows a Sample Distributor, in accordance with an embodiment of the invention.

FIG. 6 shows the system of FIG. 1 in use, in accordance with an embodiment of the invention.

### DETAILED DESCRIPTION OF SPECIFIC EMBODIMENTS

In illustrative embodiments, a Point of Care Device (PCD) and methodology for monitoring the health of an animal, such as a pet is provided. Such monitoring may track over a period of time (e.g., days, months, years) subtle changes in biomarkers, in addition to testing only when symptoms appear. The system allows a pet owner to distribute a fluidic sample across one or more electrodes, and permits the sensing of one or more target analytes which in totality can advantageously be quantitatively analyzed to provide the basis for comprehensive test results. Contamination between electrodes may advantageously limited and/or prevented. The fluidic sample tested may be saliva, which can be less invasive and often preferrable for handling and collecting compared to home urine or fecal tests. Other biological fluids may be tested, including, without limitation, urine, blood tears, interstitial fluid or any combination there. Details are discussed below.

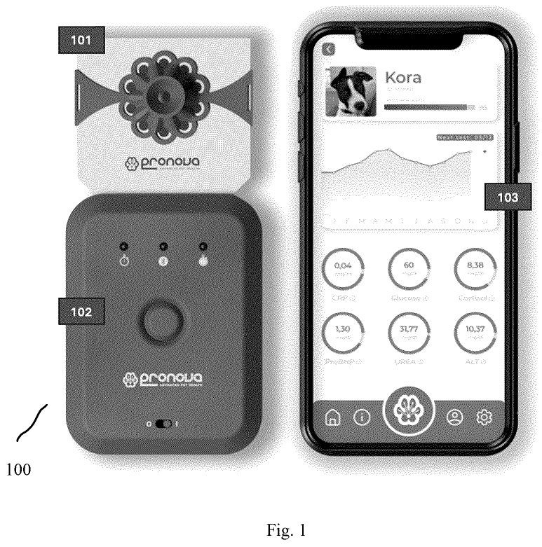

FIG. 1 shows a system 100 for monitoring the health of an animal, in accordance with an embodiment of the invention. The system 100 may be used, for example, by a pet owner to conveniently monitor the health of his pet on demand or on a periodic basis, in the comfort of their own home. Results of the test may be provided in relatively real time, without having to wait days or even weeks to get the results. For example, the results may be provided within seconds or minutes. A wide variety of animals may be tested/monitored. For example, the animal may or may not have a vertebrate. The animal may be, without limitation: a mammal, including pets (e.g., dogs, cats, ferrets, hamsters, etc.), livestock (e.g., cattle, sheep, pigs, goats, etc.), beasts of burden (horses, camels, donkeys, etc.), a wild animal (e.g., bear, cheetah, crocodile, fox, lion, shark, tiger, monkey, and leopard), birds, fish and/or amphibians. The animal may be another human being.

The system 100 may include a sensor array 101 and a Point of Care Device (PCD) 102. The system 100 may further include and/or interface with a display, a smart phone 103 and/or the Internet (or other cloud), providing access to any number of servers and associated processors/infrastructure. In some embodiments, the Point of Care Device (PCD) may be a smart phone.

More particular, the sensor array 101 may be utilized to collect a fluidic sample, and distribute it a plurality of sensing areas, each sensing area including a sensor, each sensor for sensing a same or different target analyte. The Point of Care Device (PCD) 102 may include an interface that receives input data from the sensor array 101, and a controller and/or other processor configured to analyze the input data received at the interface. The Point of Care Device (PCD) 102 may also have a user interface to control the device and/or display or otherwise provide the test results to the user. Alternatively, or in combination, the input data may be provided to a smart phone 103 and/or the Internet, where it can be, for example, stored, analyzed, and/or displayed.

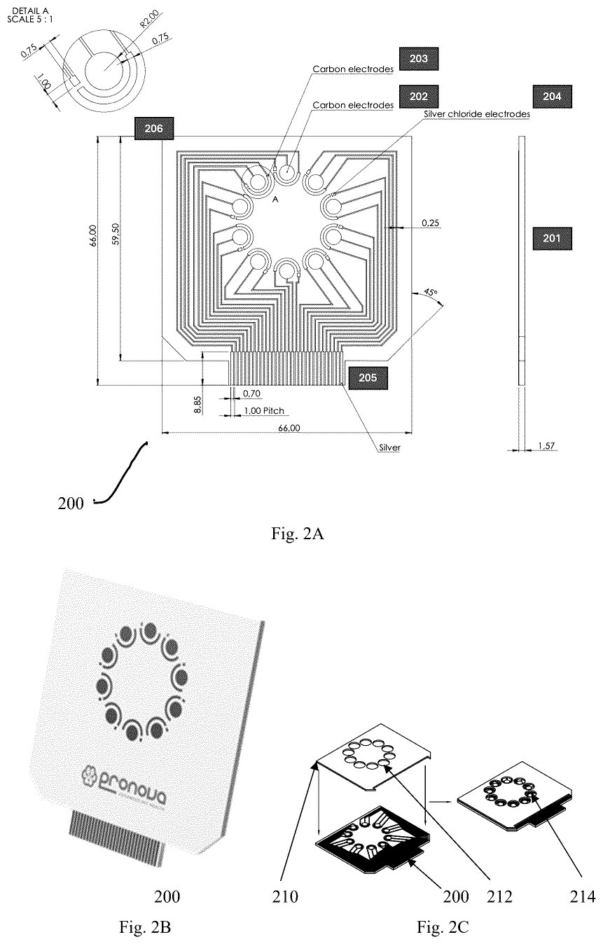

FIG. 2A shows a schematic of the sensor array 101 as, without limitation, a screen-printed electrode array 200, while FIG. 2B shows an isometric view of the screen-printed electrode array 200, in accordance with an embodiment of the invention. The screen-printed electrode array 200 may include a substrate 201, that may act as a sample strip that provides a base for a plurality of sensing areas, each sensing area including a sensor for sensing a target analyte. The substrate 201 may be made of a plastic, a paper or a ceramic, and have variable thickness. Each sensing area and/or sensor may be surrounded by walls forming a well, that is capable of storing fluidic sample. In various embodiments, the wells 214 may be fabricated by placing a cover 210 over the sample strip 200, with the cover 210 including holes 214 for positioning over the sensors/sensing areas, as shown in FIG. 2C. The walls of the holes 214 having sufficient height so that when placed over the sample strip they form wells 214.

The screen-printed electrode array 200 may include, without limitation, one or more electrodes which is printed or deposited on the substrate 201. For example, each sensor may include a working or sensing electrode 202 with a diameter of 0.1 mm to 100 mm, of variable thickness, and typically made of, but not limited to carbon or graphite, or a combination thereof.

Each working electrode 202 may be coated with a test reagent (e.g., an enzyme, antibody, aptamer and/or molecularly imprinted polymer (MIP)) that binds to a target biomarker in the sample fluid, which may be, for example, a pet's saliva. Each of the working electrodes 202 may target a different or the same analyte/biomarker. Having electrodes that target different analytes/biomarkers may advantageously allow for quantitative analysis of a health condition that would not be noticed with only one targeted analyte/biomarker.

The test reagent may bind to an analyte. The test reagent may bind to another binding reagent that binds to an analyte. The test reagent may be an analyte or an analyte analog that competes with an analyte in a fluidic sample for binding to a binding reagent for said analyte. The test reagent may bind to another binding reagent that binds to a normalization substance. The test reagent may be a normalization substance or a normalization substance analog that competes with a normalization substance in a fluidic sample for binding to a binding reagent for the normalization substance. The test reagent may be an inorganic molecule(s), an organic molecule(s) and/or a complex thereof. The organic molecule may be, without limitation, an amino acid, a peptide, a protein, a nucleoside, a nucleotide, an oligonucleotide, an oligosaccharide, a carbohydrate, a lipid, an antibody, an enzyme and/or a complex thereof. The protein may be an antigen or an antibody.

The sensor may also include: a counter electrode 203 having, without limitation, a width of 0.1 mm to 10 mm, of variable thickness, and typically made of, but not limited to carbon or graphite, or a combination thereof; and a reference electrode 204 having, without limitation, a width of 0.1 mm to 10 mm, of variable thickness, and typically made of, but not limited to silver or silver/silver chloride, or a combination thereof.

The screen-printed electrode array 200 may further include contact pads 205, where each contact pad 205 is connected to one electrode via a trace 206. The screen-printed electrode array 200 may have, for example, 2 to 90 pads, with lengths of 0.1 mm to 10 mm, and thicknesses of 0.1 mm to 10 mm, of variable thickness, and typically made of, but not limited to carbon, graphite, silver, or a combination thereof. Contact pads 205 may have a pitch (spacing between each contact pad) dictated by the pitch of the connector into which they are inserted to. The traces 206 connecting electrodes to contact pads 205 may have, without limitation, widths of 0.1 mm to 10 mm, of variable thickness, and are typically made of, but not limited to, carbon, graphite, silver, gold, or a combination thereof. Traces may have, without limitation, a spacing of 0.1 mm to 10 mm between them.

As can be seen in FIG. 2B the contact pads 205 may be positioned on an interface/connector provided on the sample strip. The connector may be, without limitation, a male or female connector, which is configured to interface with an associated connector on the Point-of-Care Device (PCD) 102. For example, the contact pads 205 on the screen-printed electrode array 200 may protrude outwards, so that they can be securely inserted into the Point-of-Care Device (PCD) 102. Also as shown in FIG. 2B, various sensors/electrodes may be left exposed so that a sample fluid can be applied. In illustrative embodiments of the invention, application of the sample fluid may be aided by a distributor, which advantageously limits and/or prevents contamination between electrodes, discussed in more detail below. In various embodiments, the sample strip that includes the sensor array 101 and/or the distributor may be a single use, disposable item.

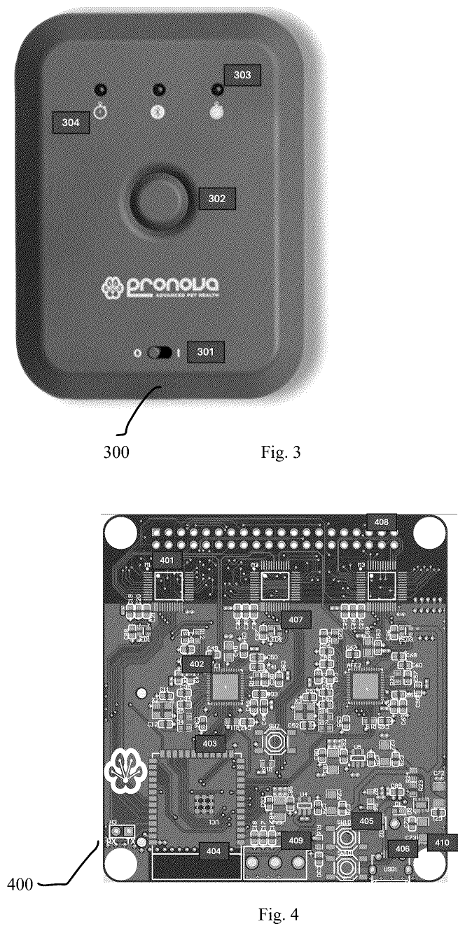

FIG. 3 shows a Point-of-Care Device (PCD) 300, in accordance with an embodiment of the invention. The Point-of-Care Device (PCD) 300 may be configured to be used at-home or in the field, and may be sized, without limitation, to be held in one's hand.

In various embodiments, the Point-of-Care Device (PCD) 300 interfaces with the sample strip/sensor array 101 to receive input data, and may serve, in part, as a multiplexor potentiostat that measures and controls the potential (or voltage) difference between various electrodes on the sensor array 101. As described above, the received input data may be analyzed at the Point of Care Device (PCD) 300. Such analysis may be done in real time. Alternatively, or in combination, the input data may be provided to a smart phone, cloud and/or the Internet, where it can be, for example, stored, analyzed, and/or displayed.

The Point-of-Care Device (PCD) 300 may include a wide variety of user interfaces. For example, the Point-of-Care Device (PCD) 300 may include a power button or switch 301, one or more trigger buttons 302 to activate and control the device and tests, one or more LEDS 303 to provide, without limitation, status, results, and error conditions, and various symbols and imagery to communicate function of the various LEDS and switches 304. In various embodiments, the Point-of-Care Device (PCD) 300 may include or interface with a display, which may be a touch panel and/or smart phone, for control, status, and to provide test results.

Within the Point-of-Care Device (PCD) 300 may be a Printed Circuit Board (PCB) 400, shown in FIG. 4, that may include and act, in part, as a multiplexer potentiostat for the analysis of the screen-printed electrode arrays or other electrochemical sensors, in accordance with an embodiment of the invention. The Printed Circuit Board (PCB) 400 may include layout for a combination of 1 or more MUX chips 401 to conduct multiplexing techniques on a range of sensors, a combination of one or more processors 402 to conduct sensing techniques, and a controller/central processing unit 403 to control Printed Circuit Board (PCB) 400 functionality.

In various embodiments, the Printed Circuit Board (PCB) 400 may also include layout for a Bluetooth and/or WiFi component 404 (or other interfaces known in the art) for external connection, control, and data transfer. In such embodiments, as opposed to the Point-of-Care Device (PCD) 300 being a standalone unit that analyzes the input data and provides test results to the user, the Point-of-Care Device (PCD) 300 may transfer the input data to a smart phone and/or the Internet/cloud, where the analysis and display of data/test results can be performed.

The Printed Circuit Board (PCB) 400 may also include layout for various user interface functions, some of which are mentioned above: one or more buttons 405 to control the device; a micro USB or other type of external connection port 406 for firmware modifications, updates, control and charging; one or more LEDs 407 for status and error presentation to user; male/female thoroughpins/ports 408 or another form of interface for connecting to sensing elements; an on-off power button 409; and/or mounting holes 410 for mounting the Printed Circuit Board (PCB) 400 onto an enclosure.

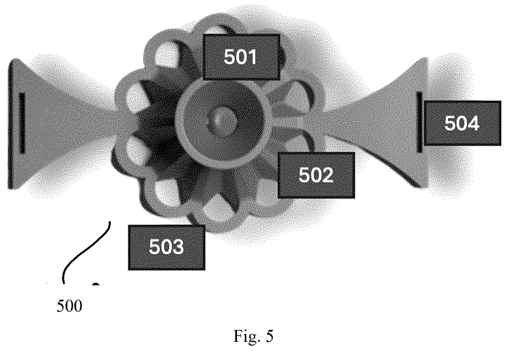

In illustrative embodiment of the invention, a sample distributor 500 may optionally be provided, as shown in FIG. 5, for receiving and distributing a sample fluid to the sensors/electrodes while limiting and/or preventing sample contamination between them. For example, and without limitation, the sample distributor may collect a sample at a central port and distribute it equally or close to equally into 2 or more partial samples to allow for multiple instances of analysis, while avoiding cross contamination by isolating each of the partial samples.

In various embodiments, the sample distributor 500 may include a central port 501 to collect the sample, where the user deposits or inserts the sample for analysis. A sloped channel 502 that utilizes gravity or microfluidic channels that use capillary action may be used to divide and direct the primary sample into secondary channels and provide the partial samples towards sensing areas such as a working electrode. The channels may include one-way valves to prevent backflow. Collection pools 503, which may or may not be covered, may allow for the partial sample to accumulate over a sensing area such as a working electrode.

The sample distributor 500 may be integrally formed with the sensor array 101/strip. In other embodiments, a clip 504 or adhesive section may secure the sample distributor to the sensor array 101/strip. The sample distributor 500 may be built with transparent, non-reactive materials allowing visual confirmation of complete filling.

In various embodiments, a hydrophobic coating may be placed between sample areas of the sensor array 101/strip to limit contamination. The coatings may be applied whether or not a sample distributor is utilized. Additionally, the use of wells to hold the sample may provide a physical barrier that assists in keeping the fluidic samples separate so as to limit contamination from other sample areas. Any combination of a sample distributor, and the use of hydrophobic coating and/or wells may be utilized.

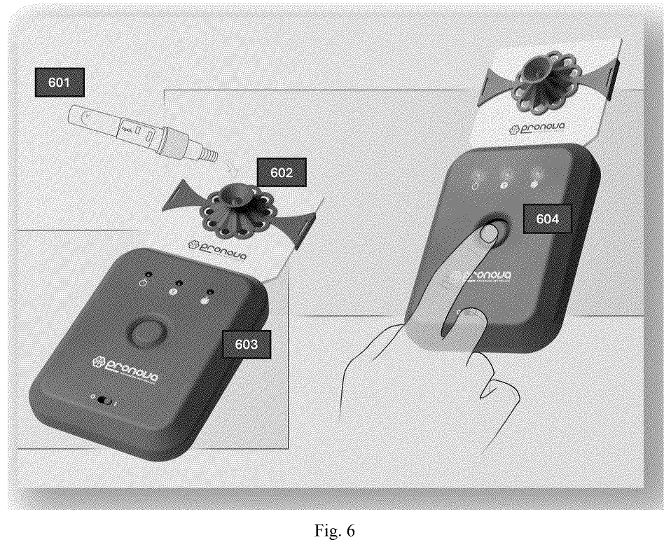

FIG. 6 shows the system of FIG. 1 in use, in accordance with an embodiment of the invention. The methodology may include, without limitation, a fluidic sample swab or collection method. Illustratively, and without limitation, the method may be a saliva swab or saliva collection method, composed of a hydrophilic material designed for rapid absorption of saliva, ensuring minimal contact time with a pet (for example), reducing stress and discomfort. The structural integrity of the swab is generally sufficient to withstand biting or chewing forces during collection. The pet owner uses the swab to collect saliva from the pet's mouth, potentially utilizing a twisting motion to ensure adequate sample collection without causing discomfort or distress.

A squeezable tube 601 may be utilized to collect the saliva from the swab. The squeezable tube 601 may be fabricated from a biocompatible, non-reactive polymer to prevent any chemical interference with the saliva sample. At a first end, the squeezable tube 601 may include a droplet tip that may be designed for precision dispensing. The squeezable tube 601 may include a pre-measured volume of a stabilizing buffer solution. In this manner, due to the potential viscosity of the saliva, the saliva sample may be pre-diluted in a buffer solution to decrease the viscosity, increase the volume, and reduce the amount of noise generated by the sample when tested. In various embodiments, the saliva sample to be tested may be pure unprocessed saliva.

The swab may be inserted directed into the squeezable tube 601. Alternatively, a syringe may be used to squeeze the saliva from the swab into the squeezable tube as follows: removing the push stick of the syringe; inserting the wet end of swab (i.e., the end which includes the saliva) into the open syringe, the wet end going in first; re-inserting the push stick back into the swab, being careful not to compress the swab yet; and then squeezing the syringe so it compresses the swab, squeezing out the saliva into the tube. Measurement indicia on the tube may be utilized so that a specific amount of saliva can be collected in the squeezable tube.

The pet owner may then squeeze and/or shake the tube 601 to mix the saliva with the buffer (if any), and extract the swab (if the syringe is not used), leaving the mixed solution in the tube 601. The solution may then be dispensed through the droplet tip onto the distributor, strip and/or sensor array 602. In this manner, a specified volume of the saliva-buffer solution is dispensed, initiating, without limitation, capillary action that spreads the solution across the electrodes, ensuring uniform exposure to the sensors/biomarkers while limiting and/or preventing cross-contamination between electrodes. The strip and/or sensor array 602 may then be inserted/connected to the Point-of-Care Device (PCD) 603 (this step may occur, without limitation, before applying the saliva buffer solution to the distributor, strip and/or sensor array 602). In various embodiments, the strip and/or sensor array 602 may be inserted into an interface device, which may then interface with the Point-of Care Device (PCD) 603 (such as a smart phone).

Instead of using a conventional swab that includes a plastic or wooden handle, in various alternative embodiments just an absorbent material may be used (e.g., a sponge-like swab without a handle), which typically may be ball or cylindrically shaped. The swab may then be inserted into a syringe to be compressed, such that the saliva is transferred into tube 106 to mix with the buffer. Advantageously, one can thus control how much saliva is provided by counting the droplets exiting the syringe. Droplets have an average size of 50 uL, so the amount of saliva collected and mixed with the buffer can be accurately estimated, and thus more accurate dilutions can be provided.

Once the sensor array is inserted into the Point-of Care Device 603 (or an interface device which then interfaces with the Point-of-Care Device 603), the user may then manipulate a user interface 604 to start the transfer of the input data and subsequent quantitative analysis/test. The controller/microprocessors within the Point-of-Care Device (PCD) 603 controls the voltage applied across the electrodes and processes the input signals (reflecting biomarker concentrations). A Bluetooth module may transmit the data to a paired mobile application, which may be a user-friendly mobile application compatible with major operating systems. The mobile application may feature secure login, pet profile management, data visualization, and educational resources. The software back-end may employ robust encryption for data security. Advanced algorithms may analyze the received data, comparing it against veterinary health standards and historical data from the pet's profile to identify trends or anomalies. Upon receiving the data from the device, the application may upload it to cloud servers for saving and performing analysis. AI-driven analytics may be performed, integrating the pet's profile data, and comprehensive health reports may be generated that may be sent back to the mobile application for user access and review. It is to be understood that in various embodiments: the Point-of-Care Device (PCD) 603 may be a standalone device which performs the analysis and includes the report generation; or the analysis may be performed, at least in part, at the mobile application without accessing the Internet and/or cloud.

Analysis of the input data may include: comparing the input signal(s) to a standard calibration curve; converting the input signal(s) signal into a concentration of said analyte; comparing concentration of said analyte to baselines and levels generated and established during clinical trials and testing; categorizing test results into a health level to inform of the animal's status and well-being; and/or recommending actions or steps formulated with veterinary experts. Analyzing a plurality of analytes may allow for additional health information to be discerned by comparing the results, concentrations and relationships between the biomarkers and their known relevance to the body's biological functions. The data analytics and machine learning may be applied to datasets of analyte concentrations and trends, along with labels data for said analytes in animals with various health conditions, to then present the probability of the pet undergoing the test being at risk of the correlated health issues.

The input data may be accrued over a period of time, such that a systematic, longitudinal analysis of the biomarkers may be performed. In this manner, subtle changes in biomarkers can be detected, allowing for, without limitation, detection of trends in the pet's health, prior to when symptoms appear.

By obtaining a plurality of different biomarkers from the sensors/electrodes (which may be advantageously aided by use of a distributor that limits and/or prevents contamination between sensors/electrodes), more comprehensive testing and analysis can be performed. Examples of utilizing two or more biomarkers to provide a health status are provided below.

Example 1: Combining multiple biomarkers can provide a comprehensive understanding of a dog's health status, especially when different biomarkers indicate related issues. Elevated CRP (C-Reactive Protein) levels may indicate inflammation, while elevated cortisol levels may suggest stress. Illustratively, elevated CRP and cortisol levels together may signal both inflammation and stress, pointing to specific health conditions such a damaged hip joint, where the inflammation from the joint issue causes elevated CRP, and the associated pain or discomfort causes elevated cortisol levels. This combination typically requires a veterinary assessment for joint damage and a pain management plan.

Example 2: Combining glucose and cortisol measurements can provide insights into the presence of Cushing's disease, a condition caused by excessive cortisol production. Elevated glucose levels may indicate potential diabetes or stress-induced hyperglycemia, while elevated cortisol levels may suggest stress or endocrine disorders. High Cortisol and ALT levels together may indicate the severity of a disease like Cushing's Syndrome.

Embodiments of the present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof.

Computer program logic implementing all or part of the functionality previously described herein may be embodied in various forms, including, but in no way limited to, a source code form, a computer executable form, and various intermediate forms (e.g., forms generated by an assembler, compiler, networker, or locator.) Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web).

Hardware logic (including programmable logic for use with a programmable logic device or, at least in part, a Point of Care Device (PCD) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL).

The embodiments of the invention described above are intended to be merely exemplary; numerous variations and modifications will be apparent to those skilled in the art. All such variations and modifications are intended to be within the scope of the present invention as defined in any appended claims. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

What is claimed is:

1. A method of monitoring health of an animal, the method comprising:
obtaining a fluidic sample from the animal;
applying the fluidic sample to a sensor array that includes a plurality of sensing areas, each sensing area including a sensor for sensing a target analyte, wherein the sensor array includes sensors that sense different target analytes;
receiving at an interface of a Point of Care Device (PCD), input data from at least two sensors from the sensor array that sense different target analytes, the Point of Care Device (PCD) including a controller configured to receive the input data from the interface;
performing quantitative analysis on the fluidic sample based on the input data; and
displaying the results of the quantitative analysis at the Point of Care Device (PCD) or at a device external to the Point of Care Device (PCD).

2. The method according to claim 1, wherein the sensor array is positioned on a sensor strip, and wherein receiving includes inserting the strip into the interface of the Point of Care Device (PCD).

3. The method according to claim 2, wherein the sensor strip is a single use, disposable item.

4. The method according to claim 1, wherein applying includes directing the fluidic sample to the plurality of sensing areas, while limiting and/or preventing contamination between the sensing areas.

5. The method according to claim 4, wherein directing includes using gravity and/or microfluidics.

6. The method according to claim 4, wherein directing includes directing the fluidic sample to the sample areas via channels, each channel including an incline and/or a one-way valve to prevent backflow.

7. The method according to claim 1, further comprising functionalizing one or more of the sensing areas and/or sensors using an enzyme, antibody, aptamer and/or molecularly imprinted polymer (MIP).

8. The method according to claim 1, further comprising mixing the obtained fluidic sample with a buffer solution prior to applying the fluidic sample to the sensor array.

9. The method according to claim 1, further comprising transmitting data associated with the input data from the Point of Care Device (PCD) to a second device and/or cloud system.

10. The method according to claim 9, wherein performing the quantitative analysis on the fluidic sample includes:
performing the quantitative analysis at the Point of Care Device (PCD), the second device and/or the cloud system; and
determining at least one health status of the animal, the health status based on data from one or more of the sensors.

11. The method according to claim 1, wherein performing the quantitative analysis on the fluidic sample is performed solely at the Point of Care Device.

12. The method according to claim 1, wherein fluidic sample is pure, unprocessed saliva.

13. The method according to claim 1, wherein performing the quantitative analysis includes applying machine learning to determine at least one health status of the animal.

14. The method according to claim 1, wherein performing the quantitative analysis includes using input data accrued over a period of time, wherein the period of time is at least one of days and years.

15. A system for monitoring health of an animal, the system comprising:
a sensor array having a plurality of sensing areas, each sensing area including a sensor for sensing a target analyte in a fluidic sample, wherein the sensor array includes sensors that sense different target analytes;
a Point of Care Device (PCD) including:
an interface that receives input data from at least two sensors from the sensor array that sense different target analytes; and
a controller configured to receive the input data at the interface and provide quantitative analysis on the fluidic sample based on the sensed target analytes.

16. The system according to claim 15 wherein the sensor array is positioned on a sensor strip, and wherein the Point of Care Device (PCD) includes a slot for receiving the strip.

17. The system according to claim 16, wherein the sensor strip is a single use, disposable item.

18. The system according to claim 15, further comprising a distributor for directing the fluidic sample to the plurality of sensing areas, while limiting and/or preventing contamination between the sensing areas.

19. The system according to claim 18, wherein directing by the distributor includes using gravity and/or microfluidics.

20. The system according to claim 19, wherein the distributor includes channels for directing the fluidic sample to particular sensing areas, each channel including an incline and/or a one-way valve to prevent backflow.

21. The system according to claim 15, wherein the controller includes a communication interface for transmitting data associated with the input data to a second device and/or cloud system, and wherein the Point of Care Device (PCD), second device and/or cloud is configured to analyze the input data and provide at least one health status, the at least one health status based on data from one or more of the sensors.

22. The system according to claim 15, wherein the controller is configured to provide the quantitative analysis utilizing input data accrued over a period of time, wherein the period of time is at least one of days and years.

23. The system according to claim 15, further including a display for displaying the results of the quantitative analysis, the display positioned on the Point of Care Device (POC) or external to the Point of Care Device (POC).
